(19) United States
(12) Patent Application Publication
(10) Pub. No.: US 2009/0202630 A1
(43) Pub. Date: Aug. 13, 2009

(54) ORALLY DISINTEGRATING TABLET COMPOSITIONS OF RANITIDINE AND METHODS OF MANUFACTURE

(76) Inventors: Gopi Venkatesh, Vandalia, OH (US); Craig Kramer, New Lebanon, OH (US); Julius Dave King, JR., Huber Heights, OH (US); Bennie L. Young, Englewood, OH (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/370,852

(22) Filed: Feb. 13, 2009

Related U.S. Application Data
(60) Provisional application No. 61/028,390, filed on Feb. 13, 2008.

(51) Int. Cl. A61K 31/341; A61K 9/50; A61K 9/20

(52) U.S. Cl. 424/464; 514/471; 424/497; 424/495; 424/494

(57) ABSTRACT

The present invention is directed to pharmaceutical compositions comprising taste-masked microcapsules comprising ranitidine, orally disintegrating tablets comprising such compositions, and methods of making the pharmaceutical compositions and dosage forms of the present invention. The present invention is also directed to methods of administering the pharmaceutical compositions and orally disintegrating tablets to treat or prevent gastrointestinal disorders.

### CROSS REFERENCE TO RELATED APPLICATIONS

This application claims the benefit of U.S. Provisional Patent Application No. 61/028,390 filed Feb. 13, 2008, the entire disclosure of which is herein incorporated by reference in its entirety for all purposes.

### BACKGROUND OF THE INVENTION

Orally disintegrating dosage forms have grown steadily in popularity as more convenient and potentially safer alternatives to conventional tablets and capsules. These rapidly disintegrating dosage forms disintegrate or dissolve in the oral cavity, and they are easily swallowed without water. They are a boon to individuals who have difficulty swallowing conventional tablets (common among geriatric and pediatric patients); people who do not have ready access to water (e.g., bed-ridden or immobile patients, or active people often away from home); and caregivers whose patients are reluctant to take their medications. Orally disintegrating dosage forms help to improve patient compliance with oral dosage regimens because they are easy to administer, convenient to take discreetly anywhere, and difficult to discard once administered.

To achieve desirable organoleptic properties, the drug particles need to be small enough and effectively taste masked and formulated into an orally disintegrating tablet (“ODT”) such that the ODT rapidly disintegrates in the oral cavity of the patient creating a smooth easy-to-swallow suspension containing taste-masked drug particles with a non-gritty mouthfeel and aftertaste. Microcapsules are small particles (typically <500 μm) encapsulated by coatings that are thick enough to prevent drug release in the oral cavity, and effectively mask the taste of underlying API.

Effective taste-masking using microcapsule technology can be challenging, especially when the API (active pharmaceutical ingredient), such as ranitidine, is extremely bitter, freely soluble in water and gastrointestinal fluids, and requires fast release in the stomach to block acid secretion. Ranitidine is a H₂-receptor antagonist indicated to treat gastric problems such as ulcers, GERD or heart burn. Ranitidine hydrochloride is freely soluble in water. It is extremely bitter with a strong aftertaste. It requires fast release in the stomach where it blocks the action of histamine on parietal cells, i.e., to inhibit acid secretion in the stomach, thus requiring fast dissolution upon entry into the stomach.

Thicker coatings required for effective taste masking of the granules may result in slower dissolution. In general, the delayed release makes it difficult to formulate an ODT that is bioequivalent to the conventional dosage form of the API. As a consequence, bitter drugs requiring rapid release in the GI tract present unique challenges in formulating orally dissolving dosage forms. Although ODTs were introduced into the market in the 1980s, these challenges have not been adequately addressed.

### SUMMARY OF THE INVENTION

In one embodiment, the present invention is directed to a taste-masked composition comprising a therapeutically effective amount of ranitidine and/or a pharmaceutically acceptable salt, polymorph, ester, or solvate thereof, as highly spherical drug particles, comprising one or more membrane layers to effectively mask the taste as well as the aftertaste of the ranitidine drug particles. In certain other embodiments, the present invention is directed to a taste-masked composition comprising a first coating comprising a water-insoluble polymeric material (e.g., applied by coacervation), and a second coating comprising a polymeric material comprising a combination of a water-insoluble polymer and a gastrosoluble pore-forming polymer (e.g., applied by fluid-bed coating). The dual coatings effectively mask the taste as well as the aftertaste of the ranitidine drug particles.

In another embodiment, the present invention is directed to an orally disintegrating tablet (ODT) composition comprising a taste-masked composition of microcapsules comprising highly spherical ranitidine drug particles comprising a therapeutically effective amount of ranitidine, a first coating comprising a water-insoluble polymeric material (e.g., applied by coacervation), and a second coating comprising a polymeric material comprising a combination of a water insoluble polymer and a gastrosoluble polymer (e.g., applied by fluid-bed coating). The ODT comprising the taste masked microparticles, rapidly dispersing microgranules, and other ODT excipients including one or more flavors, a sweetener, etc., rapidly disintegrates in the oral cavity forming a smooth, easy-to-swallow suspension exhibiting non-gritty mouthfeel and no aftertaste. These ODT tablets meet dissolution specifications of not less than 85% in 45 minutes when tested for dissolution (e.g., USP Apparatus 2 (paddles@ 50 RPM in 900 mL 0.1N HCl at 37° C.)).

In certain other embodiments, the present invention is directed to a taste-masked composition of highly spherical ranitidine drug particles, comprising a first (e.g., coacervated) polymeric membrane and a second (e.g., fluid-bed coated) polymeric membrane, and further comprising a flavor-sweetener-plasticizer layer sandwiched between the first and second membranes applied on the ranitidine drug particles. The purpose of this flavor-sweetener-plasticizer membrane is e.g., to provide temporary masking of the drug taste in case a person, especially a child consuming an ODT containing taste-masked ranitidine drug particles accidentally bites into coated drug particles. In yet another embodiment, the present invention is directed to a taste-masked composition of highly spherical ranitidine drug particles, comprising a first coacervated or fluid bed coated membrane comprising a water-insoluble polymer in combination with a gastrosoluble pore-former, and a further fluid bed coating comprising a flavor-sweetener-plasticizer layer applied over said second membrane on said ranitidine drug particles.

In other embodiments, the present invention provides methods for treating or preventing various disorders, including gastrointestinal disorders, diseases associated with the production of excess stomach acid, and anti-inflammatory disorders. In one embodiment, the methods of these embodiments comprise administering to a patient in need thereof a taste-masked composition of microcapsules comprising a drug particle comprising ranitidine, said first coacervated polymeric material comprising a water-insoluble polymer suitable for temperature-induced phase separation or microencapsulation in a solvent system, and said second fluid-bed coated blend polymeric material comprising a combination of one or more water-insoluble polymers and one or more gastrosoluble pore-forming polymers. In certain other embodiments, the present invention provides methods for a third flavor-sweetener-plasticizer layer sandwiched by fluid-bed coating between said first coacervated polymeric membrane and second fluid-bed coated blend polymeric membrane. These coatings not only effectively mask the taste/aftertaste of the ranitidine in the drug particle, but also allow fast release of ranitidine on entry into the stomach, as evident from in vitro dissolution testing in 0.1N HCl.

In another embodiment, the present invention provides a method for inhibiting a histamine H₂-receptor comprising administering to a patient in need thereof a taste-masked composition of microcapsules comprising highly spherical ranitidine hydrochloride drug particles (e.g., in polymorphic form II) comprising a first coating comprising a fluid-bed coated hydrophilic polymeric material, and a second coating comprising a coacervated polymeric material comprising a water-insoluble polymer, or a water-insoluble polymer in combination with one or more organic and/or inorganic pore formers suitable for fluid-bed coating or suitable for temperature-induced coacervation in a solvent system. These dual coatings effectively mask the taste and aftertaste of the ranitidine in the drug particle as well as provide rapid release of the ranitidine upon entry into the stomach, thereby providing a novel dosage form which is bioequivalent to the reference listed immediate release (IR) dosage form of equivalent dose.

In another embodiment, the present invention provides a method for inhibiting a histamine H₂-receptor comprising administering to a patient in need thereof a taste-masked composition of microcapsules comprising highly spherical ranitidine hydrochloride drug particles (e.g., in polymorphic form II) comprising a first coating comprising a fluid-bed coated hydrophilic polymeric material, and a second coating also comprising a fluid-bed coated polymeric material comprising one or more water-insoluble polymers, or one or more water-insoluble polymers in combination with one or more organic, inorganic, and/or polymeric pore formers suitable for processing in a fluid-bed coater. In yet another embodiment, the present invention provides a method for applying a further flavor-sweetener-plasticizer coating sandwiched by fluid-bed coating between first and second polymeric membranes, comprising appropriate amounts of one or more pharmaceutically acceptable flavoring agents, a sweetener and a plasticizer. These coatings effectively mask the taste and aftertaste of the ranitidine in the drug particle as well as provide rapid release of the ranitidine upon entry into the stomach to be bioequivalent to the reference listed immediate release (IR) dosage form of equivalent dose.

In another embodiment, the present invention is directed to a method of increasing patient compliance with a method of treating or preventing various disorders comprising administering to a patient in need thereof a therapeutically effective amount of the orally disintegrating composition as described herein, such as a composition comprising a taste-masked composition of microcapsules comprising highly spherical drug particles comprising ranitidine hydrochloride (e.g. in polymorphic form II), a first coating comprising a coacervated polymeric material, a second coating comprising a water-insoluble polymeric material and a pore-forming polymeric material, and optionally a flavor layer to protect adult or pediatric patient from experiencing drug taste in case he or she bites into coated drug particles. The various disorders include gastrointestinal disorders, diseases associated with the production of excess stomach acid, and anti-inflammatory disorders. The increase in patient compliance may be compared to a ranitidine composition that does not orally disintegrate.

In certain other embodiments, the present invention is directed to a method for the preparation of an orally disintegrating composition comprising applying onto highly spherical drug particles comprising a therapeutically effective amount of ranitidine and/or a pharmaceutically acceptable salt, polymorph, ester, or solvate thereof, with one or more taste-masking layers in accordance with the disclosures herein, preparing rapidly dispersing microgranules comprising a disintegrant and a sugar alcohol, a saccharide, or a mixture thereof; mixing said coated microcapsules, said microgranules, and ODT flavor enhancing excipients such as one or more flavoring agents, a sweetener, additional disintegrant and compression aid (e.g., microcrystalline cellulose), and optionally one or more colorants, to form a compressible blend; and compressing said compressible blend into orally disintegrating tablets of sufficient strength (e.g., about 75 mg, 150 mg or 300 mg as free base (ranitidine) to provide efficacious therapy.

These and other embodiments, advantages and features of the present invention become clear from the detailed description and examples provided in subsequent sections.

### BRIEF DESCRIPTION OF THE DRAWINGS

FIG. 1 shows micrographs of ranitidine hydrochloride drug substance with a typical particle size distribution.

FIG. 2 shows micrographs of ranitidine hydrochloride (Form II) drug substance with an average particle size of about 100 μm-400 μm and an average aspect ratio of NMT about 2-(A) API ‘as is’ and (B) After fluid-bed coating with a hydrophilic polymer.

FIG. 3 shows the dissolution profiles for orally disintegrating tablet (ODT) formulations comprising ranitidine microgranules of Example 2.

FIG. 4 shows micrographs of ranitidine hydrochloride (Form II) drug particles—FIG. 4A: Microcapsules with a first coacervated polymeric material; and FIG. 4B: Microcapsules with a first coacervated polymeric material followed by a second fluid-bed coated water insoluble—gastrosoluble polymeric blend material.

FIG. 5 shows the dissolution profiles for orally disintegrating tablet (ODT) formulation comprising ranitidine microcapsules of Example 3.

### DETAILED DESCRIPTION OF THE INVENTION

The term “drug”, “active”, “active pharmaceutical ingredient” or “API” as used herein includes a pharmaceutically acceptable and therapeutically effective compound (e.g., ranitidine), pharmaceutically acceptable salts, stereoisomers and mixtures of stereoisomers, solvates (including hydrates), polymorphs, and/or esters thereof (e.g., of ranitidine). When referring to ranitidine in the descriptions of the various embodiments of the invention, the reference also encompasses pharmaceutically acceptable salts, stereoisomers and mixtures of stereoisomers, solvates (including hydrates), polymorphs, and/or esters of ranitidine.

The terms “orally disintegrating tablet”, “orally dissolving tablet”, “orally dispersing tablet”, or “ODT” refer to a solid dosage form comprising the pharmaceutical compositions of the present invention, which disintegrates rapidly in the oral cavity of a patient after administration, without chewing. The rate of disintegration can vary but is faster than the rate of disintegration of conventional solid dosage forms or chewable solid dosage forms (i.e., tablets or capsules) which are intended to be swallowed immediately after administration. Orally disintegrating compositions of the present invention can contain pharmaceutically acceptable ingredients which swell, dissolve or otherwise facilitate the disintegration or dissolution of the ODT composition. Such ingredients can include pharmaceutical disintegrant such as crospovidone, water-soluble sugar alcohol such as mannitol, a saccharide such as lactose, or a mixture thereof, a water-soluble binder such as povidone, a meltable solid (e.g., a wax) polyethylene glycol, which can release the ranitidine upon entering the stomach. Orally disintegrating compositions of the present invention may be in the form of a tablet, a minitablet, a capsule or a monodose sachet, or a dry powder for reconstitution.

The term “about”, as used herein to refer to a numerical quantity, includes “exactly”. For example, “about 60 second” includes 60 seconds, exactly, as well as values close to 60 seconds (e.g., 50 seconds, 55 seconds, 59 seconds, 61 seconds, 65 seconds, 70 seconds, etc.).

The term “substantially masks the taste” in reference to the taste-masking layer(s) of the drug-containing core particles refers to the ability of the taste-masking layer(s) to substantially prevent release of a bitter tasting drug in the oral cavity of a patient. A taste-masking layer which “substantially masks” the taste of the drug typically releases less than about 10% of the drug in the oral cavity of the patient, in other embodiments, less than about 5%, less than about 1%, less than about 0.5%, less than about 0.1%, less than about 0.05%, less than about 0.03%, less than about 0.01% of the drug. The taste-masking properties of the taste-masking layer of the compositions of the present invention can be measured in vivo (e.g., using conventional organoleptic testing methods known in the art) or in vitro (e.g., using dissolution tests as described herein). The skilled artisan will recognize that the amount of drug release associated with a taste-masking layer than “substantially masks” the taste of a drug is not limited to the ranges expressly disclosed herein, and can vary depending on other factors, such as the perceived the bitterness of the drug and the presence of other flavoring agents in the composition.

There is provided a method for preparing an orally disintegrating tablet (ODT) composition comprising taste-masked ranitidine drug particles, rapidly-dispersing microgranules comprising a disintegrant and a sugar alcohol, a saccharide, or a mixture thereof, and other optional, pharmaceutically acceptable excipients wherein the ODT disintegrates rapidly with saliva in the buccal cavity forming a smooth, easy-to-swallow suspension. Furthermore, the ranitidine drug particles with desired particle size specifications (e.g., NMT (not more than) 5% retained on 30 mesh (>600 μm) screen and NMT 10% passing through 270 mesh (<70 μm) screen) and average aspect ratio (i.e., ratio of major axis to minor axis <about 2) comprising one or more taste-masking membranes wherein each membrane comprising a water-insoluble polymeric material, or a water-insoluble polymeric material in combination with a gastrosoluble inorganic or polymeric material, applied by solvent coacervation (e.g., temperature induced phase separation) and/or fluid bed coating, exhibit a pleasant taste/aftertaste when placed in the oral cavity and provide for rapid, substantially-complete release of the dose on entry into the stomach.

The orally disintegrating compositions of the present invention include a therapeutically effective amount of highly spherical ranitidine hydrochloride particles coated with one or more taste-masking layers, and in the form of an orally disintegrating tablet (ODT) further comprising rapidly dispersing microgranules comprising a disintegrant and a sugar alcohol, a saccharide or a mixture thereof. Upon administration of an orally disintegrating composition in an oral dosage form of the present invention (e.g., an ODT) to the oral cavity of a patient, the oral dosage form (e.g., the tablet) disintegrates rapidly in the patient's oral cavity while the rapidly dispersing microgranules dissolve into a smooth easy-to-swallow suspension containing taste-masked ranitidine HCl particles.

The rate of disintegration of orally disintegrating compositions in the oral cavity of a patient can be on the order of about 60 seconds or less, about 50 seconds or less, about 40 seconds or less, about 30 seconds or less, about 20 seconds or less, or about 10 seconds or less.

Alternatively, the rate of disintegration of the orally disintegrating compositions of the present invention can be measured using various in vitro test methods, for example the USP <701> Disintegration Test. When using the USP <701> Disintegration Test, the rate of disintegration of orally disintegrating compositions is faster than that of conventional oral, non-orally disintegrating compositions, for example about 60 seconds or less, about 30 seconds or less, about 20 seconds or less, or about 10 seconds or less. The rate of dissolution of orally disintegrating compositions of the present invention can be evaluated using the United States Pharmacopoeia Apparatus 2 (paddles@ 75 rpm in 900 mL of 0.1N HCl buffer). When using the United States Pharmacopoeia Apparatus 2 test, the rate of dissolution of the drug (e.g., ranitidine) is comparable to that of conventional, non-orally disintegrating compositions, for example about 70% or more, about 75% or more, about 80% or more, about 85% or more, about 90% or more, about 95% of the total amount of ranitidine is released in 45 min.

Abbreviations used herein include: NMT (“not more than”); FB (“fluid bed”); RLD (“reference listed drug”); and NLT (“not less than”).

The term “disposed over” means that a second material is deposited over a first material, wherein the second material may or may not be in direct physical contact with the first material. Thus it is possible, but not necessary, that an intervening material lies between the first and second materials.

The term “substantially disintegrates” means a level of disintegration amounting to disintegration of at least about 50%, at least about 60%, at least about 70%, at least about 80%, at least about 90%, or about 100% disintegration of the orally disintegrating composition.

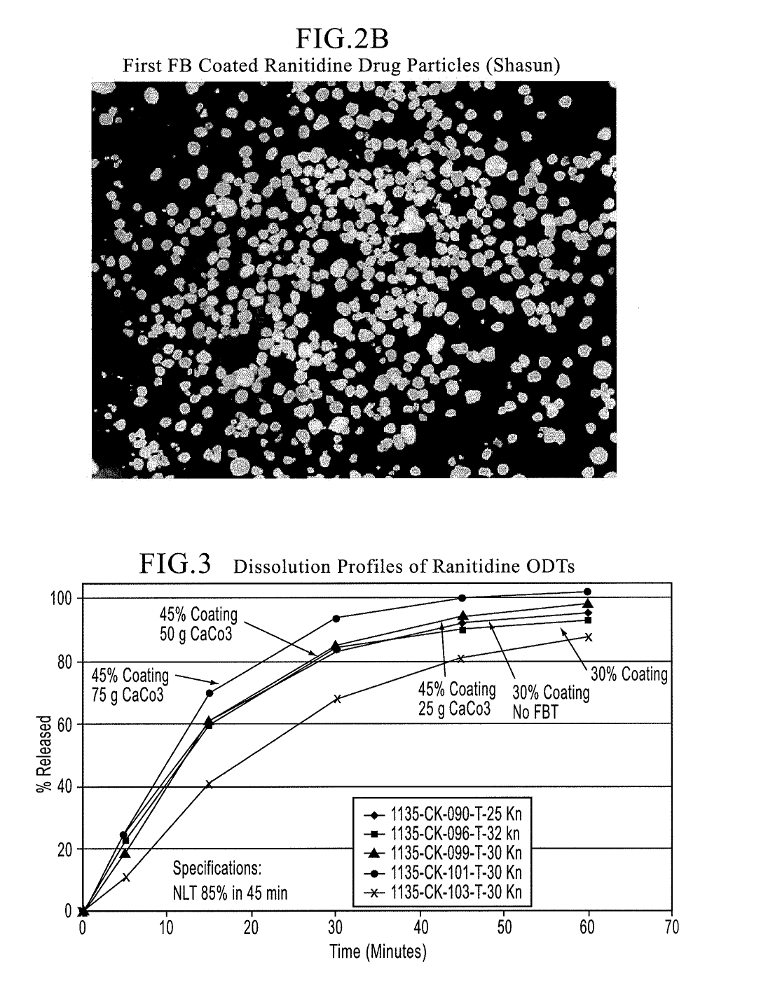

The term “ranitidine dissolution profile” refers to the dissolution profile of a ranitidine-containing composition (e.g., as shown in FIG. 3), and the plasma concentration-time profile, Cₘₐₓ, mean AUC₍₀₋₂₄₎, or AUC_{(0-inf)}, have their usual meaning. The orally disintegrating compositions of the present invention provide ranitidine release profiles which are substantially similar to that of the reference listed drug (RLD) immediate-release product (e.g., Zantac®) when tested for dissolution in 0.1N HCl. Thus, an orally disintegrating composition of the present invention will have plasma concentration-time profiles substantially similar to that of the non-orally disintegrating immediate-release ranitidine composition, and pharmacokinetics (PK) parameters, AUC₍₀₋₂₄₎ and Cₘₐₓ, may be within the 90% confidence interval (CI) of 80.0%-125.0% of the respective values for the RLD product, Zantac® dosed under identical conditions in a properly conducted crossover PK study, to be bioequivalent to the marketed product.

The term “non-orally disintegrating immediate-release ranitidine composition” refers to non-orally disintegrating compositions containing ranitidine such as conventional tablets or capsules intended to be swallowed and absorbed in the gastrointestinal tract, chewable tablets which require mastication in order to break apart the tablet structure, known in the art.

In some embodiments, a microparticle as used in the present invention refers to a particle or a granule with an average particle size of not more than about 500 μm, more particularly not more than about 400 μm. The terms “particle,” “microparticle,” “granule” and/or “microgranule” are herein used interchangeably to mean a particle with a mean particle size of not more than about 500 μm, irrespective of whether said particle contains ranitidine and/or a sugar alcohol or not. The term “microcapsule” refers to specifically taste-masked ranitidine-containing particles with a mean particle size of about 500 μm or less.

The microparticles can be described as primary particles or secondary particles. Primary particles are unagglomerated, whereas secondary particles are agglomerated primary particles. Thus, primary particles are generally smaller than secondary particles. Primary particles of ranitidine can have an average particle size ranging from about 50-500 μm, including about 75-450 μm, and about 100-400 μm and an aspect ratio (i.e., ratio of major axis to minor ratio) of not more than (NMT) about 4, including NMT about 3, and NMT about 2.

Unless indicated otherwise, all percentages and ratios are calculated by weight. Unless indicated otherwise, all percentages and ratios are calculated based on the total composition. Unless stated otherwise, the amount of the various coatings or layers described herein (the “coating weight”) is expressed as the percentage weight gain of the particles or beads provided by the dried coating, relative to the initial weight of the particles or beads prior to coating. Thus, a 10% coating weight refers to a dried coating which increases the weight of a particle by 10% after coating.

The orally disintegrating compositions of the present invention may have one or more of the following advantages: palatable ranitidine formulations with good disintegration characteristics and pharmacokinetics; improved patient compliance for patients who have difficulty swallowing conventional ranitidine tablets; and easy and/or convenient administration by the patient or the patient's caregiver.

Ideally an orally disintegrating composition should be palatable, e.g. have acceptable taste and mouthfeel characteristics. For bitter tasting drugs such as ranitidine, the orally disintegrating formulation may include a taste-masking polymer to improve the taste characteristics of the formulation, as well as a disintegrant, a sugar alcohol, a saccharide, or a mixture thereof, to provide rapid disintegration in the oral cavity as well as a “creamy” mouthfeel. In addition, the orally disintegrating formulation must also provide acceptable pharmacokinetics and bioavailability to provide the desired therapeutic effect. These desired properties of an orally disintegrating formulation can be contradictory, in that components of the formulation that provide acceptable taste-masking properties can inhibit or delay release of the ranitidine, thereby providing unacceptable pharmacokinetic properties. Conversely, components of the formulation that promote release of the ranitidine in the oral cavity result in undesirable taste or mouthfeel properties. Accordingly, an acceptable orally disintegrating formulation should balance these contradictory characteristics in order to provide a palatable (e.g., taste-masked), fast disintegrating composition with acceptable pharmacokinetics.

In one embodiment the present invention relates to a taste-masked composition comprising microcapsules comprising: (a) a drug-containing core particle comprising ranitidine and/or a pharmaceutically acceptable salt, ester, or solvate thereof, (b) a first coating comprising a coacervated polymeric material; and (c) a second coating comprising a water-insoluble polymeric material and a gastrosoluble polymer. The layers effectively mask the taste of the ranitidine and/or the pharmaceutically acceptable salt, ester, or solvate thereof.

In another embodiment, the first coating of the taste-masked composition further comprises trace amounts of a phase inducer used in the coacervation coating step, for example a commercially available polyethylene such as Epolene®.

In another embodiment, the second coating comprising a water-insoluble polymeric material and a gastrosoluble polymer further comprises one or more acetylated monoglycerides. In another embodiment, the acetylated monoglyceride derives from partially hydrogenated soybean oil. Acetylated monoglycerides are commercially available under the trade name Myvacet®.

In another embodiment, the present invention relates to a taste-masked composition comprising microcapsules comprising: (a) a drug-containing core particle comprising ranitidine and/or a pharmaceutically acceptable salt, ester, or solvate thereof; (b) a first coating comprising a hydrophilic polymeric material; and (c) a second coating comprising a water-insoluble polymeric material and a gastrosoluble pore-forming material. In another embodiment, the composition further comprises a third coating comprising one or more flavoring agents or sweeteners. The layers effectively mask the taste of the ranitidine and/or the pharmaceutically acceptable salt, ester, or solvate thereof.

In another embodiment, the present invention relates to a taste-masked composition comprising microcapsules comprising: (a) a drug particle comprising ranitidine and/or a pharmaceutically acceptable salt, ester, or solvate thereof; (b) a first coating comprising a hydrophilic polymeric material; (c) a second coating comprising a water-insoluble polymeric material and a pore-forming polymeric material; and (d) a third coating comprising one or more flavoring agents or sweeteners. The layers effectively mask the taste of the ranitidine and/or the pharmaceutically acceptable salt, ester, or solvate thereof. In another embodiment, the coatings constitute a weight gain of up to about 40%.

The compositions of the present invention can comprise any combination of a therapeutically effective amount of ranitidine, taste-masking polymers, and one or more pharmaceutically acceptable ingredients which provide an orally disintegrating composition as defined herein. For example, ranitidine hydrochloride drug substance with a desired particle size range (e.g. not more than 5% retained on 30 mesh (600 μm) screen and not more than 10% through 270 mesh screen (53 μm)) and a desired aspect ratio (ratio of major to minor axis) of NMT about 3 (i.e., highly spherical in shape) may be microencapsulated with a water-insoluble polymer by solvent coacervation in accordance with the disclosures of U.S. Pat. No. 6,139,863 and co-pending U.S. patent application Ser. No. 10/827,106 filed Apr. 19, 2004 (Publication No. U.S. 2005/0232988 published Oct. 20, 2005), the contents of which are hereby incorporated by reference in its entirety for all purposes. These taste-masked particles are combined with granules comprising a disintegrant, a sugar alcohol and/or a saccharide (granulated with purified water in a high shear granulator and dried in a tray drying conventional oven or in a fluid bed dryer material is hereafter referred to as rapidly dispersing microgranules), and compressed into an orally disintegrating tablet, whereby the disintegrant, sugar alcohol or saccharide swells and/or dissolves in the saliva of a patient's oral cavity, thereby forming a smooth easy-to-swallow suspension containing taste-masked ranitidine drug particles. Additionally, other ODT excipients such as one or more flavoring agent such as a cherry or a mint flavor, a sweetener such as sucralose, additional disintegrant (the same or a different disintegrant), and optionally one or more colorants, are included to promote rapid disintegration and to further improve organoleptic properties of the ranitidine orally disintegrating formulation.

In certain other embodiments of the present invention, the highly spherical ranitidine hydrochloride drug particles are first coated by microencapsulation by temperature-induced phase separation with a water-insoluble polymer for a weight gain of from about 10% to about 20% w/w, followed by fluid-bed coating with a water-insoluble polymer (e.g., ethylcellulose with a mean viscosity of 10 cps when tested as a 5% solution in 80% toluene/20% alcohol at ambient temperature) in combination with a gastrosoluble polymeric pore-former in accordance with the disclosure in the co-pending U.S. patent application Ser. No. 11/248,596 filed Oct. 12, 2005 (Publication No. U.S. 2006/0078614 published Apr. 13, 2006) and No. 10/521,598 filed Jul. 17, 2002 (Publication No. U.S. 2005/0269722 published Dec. 8, 2005), the contents of which are hereby incorporated by reference for all purposes.

In yet another embodiments of the present invention, the highly spherical ranitidine hydrochloride drug particles having an coating of a water-insoluble polymer by temperature-induced phase separation with for a weight gain of from about 10% to about 20% w/w and an outer coating of water-insoluble polymer (e.g., ethylcellulose with a mean viscosity of 10 cps or higher) in combination with a gastrosoluble polymer, is provided with an intermediate coating of a flavor-sweetener combination sandwiched between said first and second coatings in accordance with the present invention in order to avoid experiencing the drug taste in case of accidental biting into coated drug particles.

In one embodiment, the taste-masked microcapsules contain a sealant layer disposed over the drug-containing core particle, comprising a hydrophilic polymer. Suitable hydrophilic polymers include water-soluble polymers, polymers soluble in polar organic solvents (e.g., alcohol or acetone), or polymers soluble in a mixture of aqueous and organic solvents. Non-limiting examples of hydrophilic polymers include hydroxypropyl methylcellulose (HPMC; e.g., Opadry® Clear from Colorcon), hydroxypropylcellulose (HPC; Klucel® LF from Aqualon), and polyvinylpyrrolidone (PVP). In one embodiment, the sealant layer has a coating weight ranging from about 1% to about 10%. In another embodiment, the sealant layer has a coating weight ranging from about 0.5% to about 5%, including about 1% to about 3%, about 2% w/w.

The taste-masked microcapsules comprise one or more taste-masking layers comprising one or more water-insoluble polymers. Representative examples of water-insoluble polymers useful for taste-masking the ranitidine drug particles in accordance with the present invention include ethylcellulose, polyvinyl acetate (for example, Kollicoat SR30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Gastrosoluble agents may be polymeric or nonpolymeric, and may form pores in the coatings when used in combination with water-insoluble polymers (i.e., “pore-formers”). Nonpolymeric gastrosoluble agents may be organic or inorganic, and may be in the form of a salt. Representative examples of nonpolymeric gastrosoluble agents or pore-formers useful for taste-masking the ranitidine drug particles in accordance with the present invention include, but are not limited to, calcium carbonate, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, and the like and the mixtures thereof. The ratio of water-insoluble polymer to gastrosoluble organic or inorganic pore-former for producing taste-masked particles may typically vary from about 95/5 to about 50/50, or in some embodiments from about 85/15 to 65/35, at a thickness of from about 5% to about 50%, more particularly from about 10% to about 60% by weight of the coated drug particles.

Representative gastrosoluble polymers include but are not limited to maltodextrins, aminoalkyl methacrylate copolymers, polyvinylacetate diethaminoacetate, and combinations thereof. In one embodiment, the gastrosoluble polymer is a terpolymer based on aminoalkyl acrylate or methacrylate, butyl acrylate or methacrylate, and a methacrylate. In another embodiment, the gastrosoluble polymer is a terpolymer based on dimethylaminoethyl methacrylate, butyl methacrylate, and methyl methacrylate; and in yet another embodiment, the terpolymer has an average molecular weight of 150,000 and the ratio of the monomers is 1:2:1 of methyl methacrylate, N,N-dimethylaminoethyl methacrylate, and butyl methacrylate. An example of a gastrosoluble polymer is EUDRAGIT® E series (e.g., EUDRAGIT® E100 or EUDRAGIT® EPO). A polymer of this series has a pKa of 6.3, is soluble in gastric fluid below pH 5 while it swells and/or is permeable in water and buffer solutions above pH 5.0. The saliva is typically in the pH range of about 6.7 to 7.4. Another example of gastrosoluble polymer is poly(vinylacetal diethylaminoacetate) e.g., AEA® available from Sanlkyo Company Limited, Tokyo (Japan). The ratio of water-insoluble polymer to pore-forming polymeric material for producing taste-masked ranitidine HCl drug particles may typically vary from about 95/5 to about 50/50. The coating weight of each taste-masking coating ranges from about 5% to about 40%, by weight of the taste-masked ranitidine-containing granule, or about 5%-25%, about 5%-20%, about 5%-15%, about 5%-10%, about 10%-40%, about 10%-25%, about 10%-20%, about 10%-15%, about 15%-40%, about 50%-25%, about 15%-20%, about 20%-40%, about 20%-25%, or about 25%-40%.

The taste-masking and/or polymeric membranes described herein may include one or more plasticizers. Representative examples of plasticizers that may be used to plasticize the membranes include triacetin, tributyl citrate, triethyl citrate, acetyl tri-n-butyl citrate, diethyl phthalate, castor oil, dibutyl sebacate, acetylated monoglycerides and the like or mixtures thereof. If two or more layers of compositions described herein contain a plasticizer, the plasticizer in each plasticizer-containing layer can be the same or different. The plasticizer may comprise typically about 10-30% or about 5-15% based on the solids content of the coating formulation. In another embodiment, a coating containing a gastrosoluble polymer further comprises an anti-tack agent. Representative examples of anti-tack agents include talc, magnesium stearate and the like.

The orally disintegrating compositions of the present invention include rapidly dispersing granules comprising a disintegrant and a sugar alcohol and/or a saccharide. Non-limiting examples of suitable disintegrants for the rapidly dispersing granules can include disintegrants or so-called super-disintegrants, e.g. crospovidone (crosslinked PVP), sodium starch glycolate, crosslinked sodium carboxymethyl cellulose, low substituted hydroxypropylcellulose, and mixtures thereof. The amount of disintegrant in the rapidly dispersing granules can range from about 1%-10%, or about 5%-10% of the total weight of the rapidly dispersing granules, including all ranges and subranges therebetween.

Sugar alcohols are hydrogenated forms of carbohydrates in which the carbonyl group (i.e., aldehyde or ketone) has been reduced to a primary or secondary hydroxyl group. Non-limiting examples of suitable sugar alcohols for the rapidly dispersing granules of the orally disintegrating compositions of the present invention can include e.g. arabitol, isomalt, erythritol, glycerol, lactitol, mannitol, sorbitol, xylitol, maltitol, and mixtures thereof. The term “saccharide” is synonymous with the term “sugars” includes monosaccharides such as glucose, fructose, the lactose, and ribose; and disaccharides such as sucrose, lactose, maltose, trehalose, and cellobiose. In one embodiment, non-limiting examples of suitable saccharides for use on the compositions of the present invention can include e.g. lactose, sucrose, maltose, and mixtures thereof. In another embodiment, the rapidly dispersing granules comprise at least one disintegrant in combination with a sugar alcohol. In another embodiment, the rapidly dispersing granules comprise at least one disintegrant in combination with a saccharide. In yet another embodiment, the disintegrant-containing granules comprise at least one disintegrant in combination with a sugar alcohol and a saccharide. The amount of sugar alcohol and/or saccharide in the rapidly dispersing granules ranges from about 99%-90%, or about 95%-90% of the total weight of the disintegrant-containing granules, including all ranges and subranges therebetween. In one embodiment, the average particle size of a sugar alcohol and/or saccharide is 30 μm or less, for example about 1-30 μm, about 5-30 μm, about 5-25 μm, about 5-20 μm, about 5-15 μm, about 5-10 μm, about 10-30 μm, about 10-25 μm, about 10-20 μm, about 10-15 μm, about 15-30 μm, about 15-25 μm, about 15-20 μm, about 20-30 μm, about 20-25 μm, or about 25-30 μm.

The rapidly dispersing granules of the present invention can be prepared by any suitable method. For example, the rapidly dispersing granules can be prepared by granulation of one or more disintegrants and one or more sugar alcohols and/or saccharides in a high shear granulator, and dried in fluid bed equipment or on trays in a conventional oven to produce the rapidly dispersing granules, e.g. in the form of rapidly-dispersing microgranules. Rapidly dispersing microgranules can also be produced by the method described in U.S. Patent Application Publication No. 2005/0232988 A1, which is herein incorporated by reference in its entirety for all purposes.

In one embodiment, the compositions of the present invention contain an amount of rapidly dispersing granules and/or the mixture of a disintegrant and a sugar alcohol and/or a saccharide sufficient to provide a suitable rate of disintegration in the oral cavity of a patient forming a smooth, palatable, easy-to-swallow suspension containing ranitidine particles. The amount of a disintegrant in the rapidly dispersing granules and/or the amount of disintegrant-sugar alcohol/saccharide combination in relation to ranitidine in the compositions of the present invention can be adjusted to provide a suitable disintegration rate, as well as to form a smooth, palatable, easy-to-swallow suspension containing ranitidine particles. For example, the compositions of the present invention contain an amount of disintegrant-sugar alcohol/saccharide combination in relation to ranitidine sufficient to provide an in vitro disintegration time of about 30 seconds or less (USP <701> Disintegration Test).

The amount of rapidly dispersing granules or the amount of rapidly dispersing granules (i.e., disintegrant-sugar alcohol/saccharide combination) in relation to taste-masked ranitidine drug particles can vary depending upon the desired disintegration rate and the desired organoleptic properties including taste-masking, mouthfeel and aftertaste. The amount of rapidly dispersing granules in the compositions of the present invention can range from about 40% to about 90%, including about 40%, about 45%, about 50%, about 55%, about 60%, about 65%, about 70%, about 75%, about 80%, and about 85%, inclusive of all values, ranges, and subranges therebetween. In one embodiment, the amount of rapidly dispersing granules is about 60-70% of the total weight of the composition. In another embodiment, the rapidly dispersing granules is about 65% by weight.

Likewise, the compositions of the present invention should contain a sufficient quantity of taste-masked ranitidine drug particles to provide a therapeutically effective dose of ranitidine. The amount of taste-masked ranitidine drug particles in the orally disintegrating compositions of the present invention can be adjusted to provide a therapeutically effective dose of ranitidine.

The amount of ranitidine hydrochloride in the orally disintegrating compositions of the present invention can range from about 5% to about 50%, including about 5%, about 10%, about 15%, about 20%, about 25%, about 30%, about 35%, about 40%, about 45%, and about 50%, inclusive of all values, ranges, and subranges therebetween. In one embodiment, the amount of taste-masked ranitidine drug particles in the orally disintegrating compositions of the present invention is about 30% by weight of the orally disintegrating composition.

In some embodiments, the compositions of the present invention can comprise highly spherical ranitidine hydrochloride drug particles (e.g., crystals), coated with two or more taste-masking layers. The taste-masking layer(s) (as described herein) and additional coatings can be applied onto highly spherical ranitidine hydrochloride drug particles by any suitable combination of taste-masking methods, for example (1) coacervation followed by fluid bed coating, (2) fluid bed coating followed by coacervation, (3) coacervation followed by two successive fluid bed coating, and (4) fluid bed coating followed by coacervation followed by fluid bed coating.

The compositions of the present invention may further comprise one or more pharmaceutically acceptable, flavoring agents. Non-limiting examples of such flavoring agents include, for example, cherry, spearmint, orange, or other acceptable fruit flavors, or mixtures of cherry, spearmint, orange, and other acceptable fruit flavors, at up to about 5% based on the tablet weight. In addition, the compositions of the present invention is can also include one or more sweeteners such as aspartame, sucralose, or other pharmaceutically acceptable sweeteners, or mixtures of such sweeteners, at up to about 2% by weight, based on the tablet weight. Furthermore, the compositions of the present invention can include one or more FD&C colorants at up to about 0.2% to 2% by weight, based on the tablet weight.

The compositions of the present invention can also include an additional disintegrant, in addition to the disintegrant in the rapidly dispersing granules (e.g., ranitidine containing and/or rapidly-dispersing granules). The additional disintegrant can be the same disintegrant used in the disintegrant-containing granules, or a different disintegrant. The additional disintegrant may be present in the compositions of the present invention at up to about 10% based on the tablet weight.

The compositions of the present invention can also include a pharmaceutically acceptable microcrystalline cellulose, e.g. Avicel PH101, Avicel PH102, Ceolus KG-802, Ceolus KG-1000, Prosolv SMCC 50 or SMCC90 or other pharmaceutically acceptable grades of microcrystalline cellulose, as well as mixtures thereof.

In one embodiment, the orally disintegrating compositions of the present invention comprise about 25-35% of ranitidine HCl drug particles, microencapsulated with a coacervated taste-masking layer comprising a water-insoluble polymer (e.g., ethylcellulose), about 60-70% of rapidly-dispersing granules (e.g., comprising crospovidone and mannitol); about 5% of additional disintegrant (e.g., crospovidone); about 5% to 15% by weight of microcrystalline cellulose, about 0.5-2.0% of one or more flavors, and about 0.5%-1% of a sweetener (e.g., sucralose).

In accordance with certain embodiments of the present invention, the method may include the steps of
i. taste-masking highly spherical ranitidine HCl drug particles by (a) fluid bed coating with a hydrophilic polymer (e.g., hypromellose as Opadry® Clear from Colorcon) for a weight gain of from about 1% w/w to 10% w/w, and (b) solvent coacervation with a water-insoluble polymer (e.g., ethylcellulose with a mean viscosity of 100 cps) alone for a weight gain of from about 20% w/w to about 40% w/w, (c) solvent coacervation with a water-insoluble polymer (e.g., ethylcellulose) in combination with a gastrosoluble pore-former (e.g., calcium carbonate) in accordance with the disclosure in the co-pending U.S. patent application Ser. No. 11/213,266 filed Aug. 26, 2005 (Publication No. U.S. 2006/0105038 published May 18, 2006), the contents of which are incorporated by reference for all purposes.
ii. granulating a powder mixture of a sugar alcohol such as mannitol or a saccharide such as lactose and crospovidone, for example, using the disclosures in EP 0914818 and the co-pending U.S. patent application Ser. No. 10/827,106 filed Apr. 19, 2004 (Publication No. U.S. 2005/0232988 published Oct. 20, 2005), the contents of which are hereby incorporated by reference to produce rapidly-dispersing microgranules;
iii. blending appropriate amounts of the taste-masked drug particles from step (i), rapidly-dispersing microgranules from step (ii) and other pharmaceutically acceptable excipients; and
iv. compressing the blend from step (iii) into orally disintegrating tablets in accordance with the disclosures of U.S. Pat. No. 6,964,779 and U.S. Pat. No. 5,700,492 each of which is incorporated by reference in its entirety, comprising required doses of Ranitidine HCl ODTs, 75-mg and 150-mg (equivalent ranitidine base) which would rapidly disintegrate on contact with saliva in the buccal cavity forming a smooth, easy-to-swallow suspension and exhibit pharmacokinetics parameters in the range of 80.0% to 125.0 of 90% confidence interval.

One embodiment of a method for the preparation of a taste-masked compositions of the present invention comprises:
a. a first coating step comprising coating highly spherical drug particles of ranitidine hydrochloride and/or a pharmaceutically acceptable salt, solvate, or ester thereof with a first coating, comprising a water-insoluble polymer, applied by phase-induced solvent coacervation to form coated drug particles; and
b. a second coating step comprising coating said coated drug particles with a second polymeric coating material comprising a water-insoluble polymer and a gastrosoluble polymer to form coated microcapsules.
   The polymeric materials can comprise about 10% to about 20% by weight of said first coating on said microcapsules.

In accordance with certain embodiments of the present invention, the method may include the steps of
i. taste-masking highly spherical ranitidine HCl drug particles by (a) solvent coacervation with a water-insoluble polymer (e.g., ethylcellulose with a mean viscosity of 40 cps or higher) for a weight gain of from about 10%-20% w/w followed by (b) an outer coating in a fluid bed coater with a water-insoluble polymer (e.g., ethylcellulose with a mean viscosity of 7 cps or higher) in combination with a gastrosoluble polymer (e.g., Eudragit EPO/E100) at a ratio of 90/10 to about 50/50 for a weight gain of 20% to 40% by weight, in accordance with the disclosure in the co-pending U.S. patent application Ser. No. 11/248,596 filed Oct. 12, 2005 (Publication No. U.S. 2006/0078614 published Apr. 13, 2006), the contents of which are hereby incorporated by reference for all purposes;
ii. granulating a powder mixture of a sugar alcohol such as mannitol or a saccharide such as lactose and a super disintegrant such as crospovidone, to produce rapidly-dispersing microgranules;
V. blending appropriate amounts of the taste-masked drug particles from step (i), rapidly-dispersing microgranules from step (ii) and other pharmaceutically acceptable excipients such as one or more flavoring agents, colorants, a sweetener, a diluent (compression aid) such as microcrystalline cellulose, and additional disintegrant; and
iii. compressing the blend from step (iii) into tablets comprising required doses of Ranitidine HCl ODTs, 75-mg and 150-mg (equivalent ranitidine base) which would rapidly disintegrate on contact with saliva in the buccal cavity forming a smooth, easy-to-swallow suspension and exhibit pharmacokinetics parameters in the range of 80.0% to 125.0% of 90% confidence interval

In accordance with certain embodiments of the present invention, the method of taste-masking of highly spherical ranitidine hydrochloride (Form II) drug particles may include the steps of (a) a first coating comprising a coacervated polymeric material, (b) an intermediate flavor-sweetener coating comprising one or more flavoring agents, one or more colorants, a sweetener, a plasticizer, and optionally a film forming polymeric binder, and (c) a second fluid-bed coated water insoluble-gastrosoluble polymeric blend material.

In yet another embodiment, the method includes the steps of applying onto ranitidine drug particles in a fluid-bed coater first hydrophilic polymeric membrane for a weight gain of from about 0.5% to about 5% w/w comprising a water-soluble polymer and a second membrane also applied in a fluid bed coater for a total weight gain of up to about 40% w/w comprising a water-insoluble polymer in combination with a gastrosoluble polymer (e.g., as a pore former).

In one embodiment, the first coating step comprises (i) mixing a water-soluble polymer with a polar and/or nonpolar organic solvent mixture to dissolve said polymer and applying the coating onto said highly spherical drug particles while maintaining said drug particles at a desired fluidized product bed temperature, and said second coating step involves (i) mixing said first coated drug particles with a first water-insoluble polymer (ethylcellulose) and a nonpolar organic solvent (cyclohexane) and a phase inducer (polyethylene wax) to form said drug particle-polymer mixture; (ii) heating said drug particle-polymer mixture at a first temperature so that said first polymeric material dissolves in said nonpolar organic solvent; (iii) cooling said drug particle—polymer mixture over time while stirring to a second temperature to form a dispersion of coated drug particles; (iv) recovering said coated drug particles; and (v) drying said coated drug particles. In another embodiment, an additional step of applying a flavor-sweetener coating composition onto said coacervated first polymer coated drug particles in fluid bed coater may be included in the manufacturing process.

In another embodiment, both first and second coating steps applied in a fluid-bed coater involve the first membrane applied for a gain of from about 1% to about 10% w/w comprising a hydrophilic polymer, and the second membrane applied for a total weight gain of up to about 40% by weight of the coated drug particle comprising a water-insoluble polymer in combination with a gastrosoluble pore forming polymer. In yet another embodiment, an additional step of applying a flavor-sweetener coating composition onto said first polymer coated drug particles in fluid bed coater may be included in the total manufacturing process.

In one embodiment, the first coating step comprises (i) mixing said drug particle, a nonpolar organic solvent, a phase inducer such as polyethylene wax, and said first polymeric coating material to form a drug-particle mixture; (ii) heating said drug-particle mixture at a first temperature so that said first polymeric material dissolves in said nonpolar organic solvent; (iii) cooling said drug core mixture over time while stirring to a second temperature to form a dispersion of coated drug particles; (iv) recovering said coated drug particles; and (v) drying said coated drug particles, and the second coating step comprises (i) mixing a water insoluble polymer (ethylcellulose), gastrosoluble polymer (Eudragit E100), a plasticizer (triethyl citrate) with a nonpolar solvent to dissolve, (ii) homogeneously suspending an anti-tack agent (talc or magnesium stearate) and (iii) spraying onto singly coated drug particles while maintaining said singly coated drug particles at a desired product temperature and in an appropriately fluidized state to avoid agglomeration of said drug particles, and (iv) drying the coated drug particles. In another embodiment, an additional step of step of applying a flavor-sweetener coating composition onto said first polymer coated drug particles in fluid bed coater may be included in the total manufacturing process.

One embodiment of a method for producing pleasant tasting ranitidine orally disintegrating formulations of the present invention, comprising ranitidine hydrochloride microparticles with a mean particle size of about 100-400 μm and a mean aspect ratio of NMT about 3 (e.g., highly spherically particles with a mean particle size of about 100-400 μm), comprises (i) taste-masked drug particles, (ii) preparing rapidly dispersing granules comprising a disintegrant, a sugar alcohol and/or a saccharide, and (iii) forming the oral dosage form. The step of forming the oral dosage form can comprise, for example, compressing a blend comprising said taste-masked ranitidine microparticles and said rapidly dispersing granules, optionally with pharmaceutically acceptable flavorant(s), sweetener(s), other disintegrant(s), colorant(s) and/or compression aides such as microcrystalline cellulose in sufficient quantities into the orally disintegrating form using a tablet press, such as a rotary tablet press equipped with an external lubrication system to lubricate the punches and dies prior to compression. These orally disintegrating tablets rapidly disintegrate upon exposure to the saliva in the mouth into a smooth, easy-to-swallow suspension with no gritty aftertaste.

In another embodiment, the method for preparing orally disintegrating formulations of the present invention comprising ranitidine HCl microparticles with a mean particle size of about 100-400 μm includes at least a two-step process for taste-masking said drug particles by coacervation followed by fluid bed coating as described above, prior to blending and compression into orally disintegrating tablets. In yet another embodiment, the method for preparing orally disintegrating formulations of the present invention comprising highly spherical ranitidine hydrochloride drug particles with a mean particle size of about 100-400 μm includes a three-step process for taste-masking said drug particles by coacervation followed by an intermediate coating with a flavor-sweetener combination and an outer coating with a water-insoluble polymer in combination with a gastrosoluble polymeric pore-former, the latter two membranes being applied in a fluid bed coater as described above, prior to blending and compression into orally disintegrating tablets.

In another embodiment, the method of preparing the compositions of the present invention includes a taste-masking step. The taste-masked ranitidine drug particles of the compositions of the present invention (e.g., highly spherical ranitidine hydrochloride particles) of the present invention can be prepared by various methods, including solvent coacervation with a water-insoluble polymer such as ethylcellulose. In one embodiment, the water-insoluble polymer (e.g., ethylcellulose), a phase-inducer (e.g., polyethylene), and ranitidine drug particles are loaded into a coacervation tank containing cyclohexane. The mixture in the tank is heated to about 80° C. to dissolve the ethylcellulose, and then slowly cooled under controlled conditions thereby causing phase-induced microencapsulation of ranitidine particles by the ethylcellulose. Microencapsulation or coacervation refers to the process of applying a membrane by phase separation for imparting taste-masking (or sustained release) properties. Upon reaching ambient temperature, the suspension of microencapsulated ranitidine particles are filtered, washed with fresh cyclohexane and dried to reduce residual solvent levels within acceptable limits (e.g., <4,000 ppm), in one embodiment less than 1,000 ppm. The coating weight of the microencapsulated ranitidine particles can range from about 5% to about 45% including about 10%, 15%, 20%, 25%, 30%, 35%, 40%, and 45%, inclusive of all ranges and subranges therebetween. Examples of such a coacervation process are disclosed in U.S. Pat. No. 6,139,865.

Alternatively, the coacervation solution can comprise a mixture of the water-insoluble polymer (e.g., ethylcellulose) and a water-insoluble or gastrosoluble pore-former (e.g., calcium carbonate). The ratio of water-insoluble polymer to pore-former can range from about 50/50 to 95/05, including about 55/45, about 60/40, about 65/35, about 70/30, about 75/25, about 80/20, about 85/15, and about 90/10, including all ranges and subranges therebetween. The coating weight of the microencapsulated ranitidine particles can range from about 5% to about 40% including about 10%, 15%, 20%, 25%, 30%, and 35% inclusive of all ranges and subranges therebetween. In one embodiment, the coacervation step comprises suspending the drug-containing particles in a solution of water-insoluble ethylcellulose at 80° C. in the coacervation tank. During the cooling cycle, the micronized pore-former is introduced into the tank at a temperature of about 58° C., while constantly stirring the suspension to uniformly distribute the pore-former in the microcapsule-membrane, at the forming/hardening phase. Examples of such a coacervation process are disclosed in U.S. Patent Application Publication No. 2006/0105038 A1, which is herein incorporated in its entirety by reference for all purposes.

Microencapsulation by coacervation provides properties that are desirable for taste-mask coatings. Compared to other coating techniques, coacervation provides coatings of uniform thickness and creates an effective barrier around the bitter drug at low coating levels even when the drug particles are small.

In another embodiment, the methods of the invention includes steps to prepare orally disintegrating tablets by mixing ranitidine microgranules or taste-masked ranitidine microparticles, one or more flavoring agents, a sweetener, rapidly-dispersing microgranules, microcrystalline cellulose, additional disintegrant, and magnesium stearate and compressing this mixture into orally disintegrating tablets using a conventional rotary tablet press. The orally disintegrating tablets formed thereby may provide: rapid disintegration on contact with saliva in the buccal cavity, a pleasant taste (good creamy mouth feel), and/or rapid, substantially-complete release of the dose in the stomach.

In yet another embodiment, the methods of the invention include steps to prepare an orally disintegrating tablet formed by compressing a composition comprising ranitidine-containing particles, rapidly-dispersing granules, and optionally flavoring agents, sweeteners, and other pharmaceutically acceptable excipients in a tablet press equipped with an externally lubricating system to pre-lubricate dies and punches, thereby providing a tablet formulation otherwise free of lubricant. The orally disintegrating tablets thus produced typically exhibit sufficient hardness and sufficiently low friability to be suitable for packaging in HDPE bottles and push-through film backed or peel-off paper backed blister packs using conventional equipment for storage, transportation and commercial distribution.

The methods of the invention produce pharmaceutical compositions (e.g., orally disintegrating compositions comprising ranitidine taste-masked microparticles as described herein) that provide acceptable taste-masking when placed in the mouth until swallowed (e.g., not more than about 10% of the ranitidine dose released in about 3 minutes when tested for dissolution in simulated saliva fluid at pH of about 7.0). In another embodiment, the orally disintegrating dosage form will disintegrate in about 30 seconds when evaluated using the USP <701> Disintegration Test. The orally disintegrating dosage form will typically disintegrate on contact with saliva in the buccal cavity in about 60 seconds, forming a smooth, easy-to swallow suspension of taste-masked microparticles with an acceptable aftertaste. These taste-masked microparticles will typically provide substantially complete release of the ranitidine dose upon entering the stomach (e.g., not less than about 60%, more particularly not less than 70% of the dose released in about 30 minutes when tested for dissolution in simulated gastric fluid or 0.01N HCl).

In another embodiment, a method of manufacturing orally disintegrating compositions of the present invention comprise the following steps:

a first coating step comprising coating drug particles of ranitidine and/or a pharmaceutically acceptable salt, solvate, or ester thereof with a first polymeric coating material to form coated drug particles; and

a second coating step comprising coating said coated drug particles with a second polymeric coating material comprising a water-insoluble polymer and a pore-forming polymer to form coated microcapsules.

In another embodiment, a method of manufacturing orally disintegrating compositions of the present invention comprises the following steps:

coating drug particles comprising ranitidine and/or a pharmaceutically acceptable salt, ester, or solvate thereof with one or more taste-masking layers to form coated microcapsules;

preparing granules comprising (i) a disintegrant and (ii) a sugar alcohol, a saccharide, or a mixture thereof;

mixing said coated microcapsules; and

forming the final dosage form.

In Vitro Disintegration Time/Dissolution Testing

Disintegration times are measured using the USP <701> Disintegration Test procedures. The taste-masking property of the taste-masked microparticles and the orally disintegrating tablets may be evaluated in the mouth by determining the percentage of drug-release when tested for dissolution using USP Apparatus 2 (paddles @ 75 rpm) in 900 mL of saliva-simulating fluid at a pH of about 6.8-7.0 (a release of not more than about 10% of the dose in about 3 minutes is considered acceptable). In addition, the rapid-release property in the stomach of the taste-masked microparticles and the orally disintegrating tablets may be evaluated by determining the percentage of drug-release when tested for dissolution using USP Apparatus 2 (paddles @ 75 rpm) in 900 mL of 0.01N HCl at 37.0±0.5° C. (a release of not less than about 70% of the dose in about 30 minutes is considered acceptable). The potency of the tablets and the percentage of drug dissolved at different time points are determined using a validated HPLC methodology using a Waters XBridgeShield RP18 3.5 μm, 100×4.6 mm (or equivalent column) or an alternative suitably validated methodology.

In their various embodiments, the orally disintegrating compositions of the present invention comprising ranitidine microparticles exhibit one or more of the following properties:

acceptable hardness and friability suitable for packaging in bottles and blister packaging, storage, transportation and commercial distribution;

disintegration on contact with saliva in the oral cavity in about 60 seconds forming a smooth, easy-to-swallow suspension with a pleasant taste (no grittiness or aftertaste), meeting the specification of not more than 30 seconds in the <USP 701> Disintegration Test;

taste-masked ranitidine drug particles exhibit smooth mouthfeel (non-gritty) and no aftertaste; and

provide rapid, substantially-complete release of the dose upon entry into the stomach, as evident by meeting the dissolution specifications of about 85% of the dose in about 45 minutes in 900 mL of 0.01N HCl buffer when tested for dissolution using USP Apparatus 2 (paddles @ 75 rpm).

The compositions of the present invention are useful in treating or preventing gastrointestinal disorders, diseases associated with the production of excess stomach acid, and/or inflammatory disorders. The compositions may also be useful in inhibiting a histamine H₂-receptor. They can contain a therapeutically effective amount of ranitidine. The compositions of the present invention can comprise about 1-250 mg of ranitidine, including about 2 mg, 5 mg, 25 mg, 50 mg, 100 mg, 150 mg, and 200 mg of ranitidine. The compositions of the present invention can be administered according to any suitable dosage schedule as can be readily determined by a physician. For example, the compositions of the present invention can be administered in a single daily dose, or multiple daily doses, depending, for example, upon the severity of the condition and physical condition of the patient.

The following non-limiting examples illustrate the compositions of the present invention, comprising microgranules, taste-masked microparticles or the orally disintegrating tablet dosage form, wherein the composition comprising ranitidine, taste-masked or otherwise. The compositions of the present invention are prepared as described herein, and exhibit acceptable organoleptic properties when placed in the mouth and substantially complete, rapid release of the dose on entry into the stomach.

### Example 1

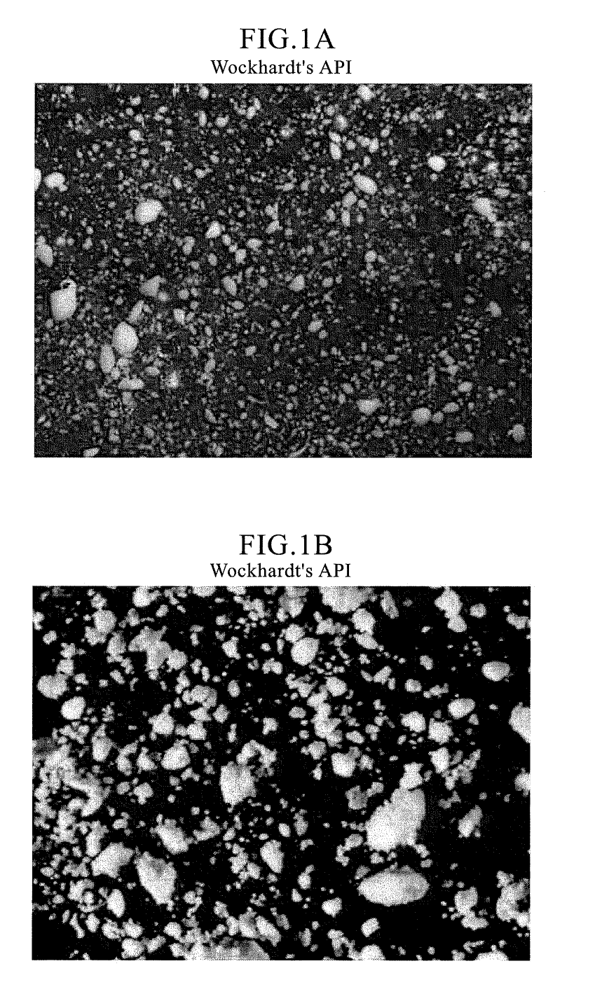

As is apparent from FIG. 1 that shows the micrographs of Ranitidine hydrochloride (form II) drug substance from two of many API manufacturers, the drug substance has typically a significantly wider particle size distribution, as well as a wider aspect ratio (ratio of major axis to minor axis). Consequently, it is difficult to effectively taste-mask these drug particles by coacervation, and/or by fluid-bed coating and incorporate the microcapsules thus obtained into an ODT (orally disintegrating tablet) expecting it to disintegrate on contact with the saliva in the oral cavity into a smooth easy-to-swallow suspension with a non-gritty mouthfeel and no aftertaste.

1.A Ranitidine hydrochloride microgranules: 49 parts of ranitidine hydrochloride drug substance with a mean particle size of 12-16 μm (Polymorph Form II from Vera Laboratories) was blended with 45 parts of mannitol and 5 parts of hypromellose (Methocel Premium 400 cps from Dow Chemicals) in a high shear granulator, KG 5, granulated with a solution (15% solids) of hypromellose (Methocel E5 from Dow Chemicals) as the binder, wet milled and dried in a tray drying oven for a loss on drying (LOD) of <1% by weight.

1.B Microencapsulation of Granules: Microgranules (73.3 g) with a low friability obtained above were microencapsulated using the solvent coacervation process. Ethocel (ethylcellulose) Standard 100 Premium (100 cps), from Dow Chemicals (60 g) and Epolene (polyethylene wax with a weight averaged molecular weight of 6000) were charged into a 4L coacervation tank containing 2000 g of cyclohexane. The ethylcellulose was dissolved by subjecting the coacervation tank to a ‘heat to 80° C. plus hold’ routine with the agitator (mixing speed) at 150 RPM. Thereafter, the tank was cooled at a rate of about 1° C. per min. Upon reaching a temperature of 80° C., the agitator speed was increased to 300 RPM and cooling continued to below 30° C. Upon reaching the ambient temperature, the microcapsules with a membrane coating of approximately 45% by weight were filtered, washed with fresh cyclohexane and dried in the hood to reduce the residual solvent level to <2000 ppm. The taste-masked microparticles with more than 80% of the microcapsules in the range of <30-80>mesh (250-600 μm) had acceptable taste-masking.

TABLE 1
Compositions of Microcapsules and ODTs of Ranitidine HCl

| | Microencapsulation - Quantity per Batch (g) | | | | |
|---|---|---|---|---|---|
| Ingredient | Formula 1 | Formula 2 | Formula 3 | Formula 4 | Formula 5 |
| Ranitidine HCl | 73.3* | 73.3 | 73.3 | 93.3 | 48.9 |
| Ethylcellulose | 60.0 | 60.0 | 60.0 | 40.0 | 40.0 |
| Polyethylene | 30.0 | 25.0 | 25.0 | 40 | 40.0 |
| Ca Carbonate | | | 20.0 | 8.0 | |
| | Orally Disintegrating Tablets - % per Tablet | | | | |
| Reference # | 966-155** | 1135-005 | 921-190 | 1135-050 | 1135-027 |
| Microcapsules | | 33.24 | 33.24 | 33.00 | 33.00 |
| RD Granules | | 60.98 | 60.98 | 60.35 | 60.38 |
| Aspartame | | 0.35 | 0.35 | 0.0 | 0.0 |
| Sucralose | | 0.0 | 0.0 | 0.15 | 0.15 |
| Strawberry Flavor | | 0.43 | 0.43 | 0.0 | 0.0 |
| Cherry Flavor | | | | 0.50 | 0.50 |
| Crospovidone XL-10 | | 5.00 | 5.00 | 5.00 | 5.00 |
| Citric Acid | | 0.0 | 0.0 | 0.50 | 0.50 |
| FD&C Red No 40 | | 0.0 | 0.0 | 0.50 | 0.50 |
| Tablet Weight (mg) | | 919.0 mg | 1125.8 mg | 1029.6 mg | 731.6 mg |
| Polymer Coating | | 45% | 45% | 45% | 30% |
| Polymer:Pore former | | | 2:1 | 5:1 | |
| Taste-masking Rating^{♦} | 9 | 7 | 4 | 8 | 8 |
| % Dissolved^{†} at: | | | | | |
| 15 min | 13 | 47 | 81 | 44 | 52 |
| 30 min | 40 | 66 | 97 | 68 | 71 |
| 45 min | 51 | 77 | 107 | 81 | 81 |

*Ranitidine microgranules of Ex. 1.A.
**Not tableted. Microcapsules tested for dissolution.
^{♦}Taste-masking rating: 1 (poor) → 5 (pleasant) → 10 (excellent)
^{†}Dissolution: USP Apparatus 2 (Paddle@ 100 rpm, 900 ml Water at 37° C.)

1.C Rapidly Dispersing Granules: The rapidly dispersing microgranules comprise a sugar alcohol such as mannitol and/or a saccharide such as lactose and a disintegrant such as Crospovidone. The sugar alcohol and/or saccharide and disintegrant will typically be present in the rapidly dispersing microgranules at a ratio of from about 99:1 to about 90:10 (sugar alcohol and/or saccharide:disintegrant). For example, D-mannitol, a sugar alcohol with an average particle size of about 15 μm and Crospovidone XL-10, a super disintegrant, at a ratio of about 95/5 were granulated in a high shear granulator using purified water as the granulating fluid, wet milled, dried in a tray drying oven for an LOD of less than 1% by weight, and dry milled to produce rapidly dispersing granules with an average particle size of about 200-400 μm.

1.D Orally Disintegrating Tablets of Taste-masked Granules: The taste-masked ranitidine granules, rapidly-dispersing microgranules, pre-blended mix comprising crospovidone, strawberry flavor, aspartame (a sweetener), were blended together and compressed using a Carver press to produce ODTs weighing 1 g, containing 75 mg of equivalent ranitidine base (see Table 1 for composition and dissolution data of the ODT). Although the ODTs had acceptable taste-masking, these tablets had a gritty mouthfeel and poor dissolution profile.

### Example 2

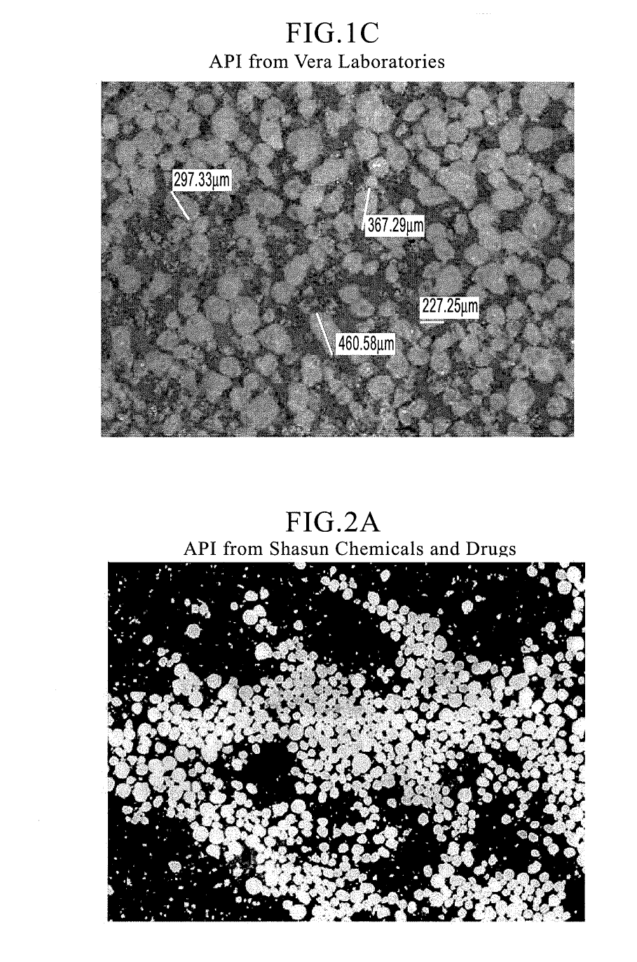

2.A Microcapsules of Ranitidine Hydrochloride: Ranitidine hydrochloride (Form II from Shasun Drugs and Chemicals) with desired particle size specifications (e.g., NMT 5% retained on 35 mesh (500 μm) and NMT 10% passing through 270 mesh (70 μm)) and average aspect ratio specification of NMT about 3 (see FIG. 2A for micrographs of the drug substance), Ethocel Premium and Epolene were charged into the 4L coacervation tank (see Table 1 for compositions of Microcapsules lots #1135-005 at 45% ethylcellulose coating and 1135-027 at 30% ethylcellulose coating) and were taste-masked in accordance with the disclosures of Example 1.B above. During the microencapsulation of Microcapsules lots# 921-190 and 1135-050, micronized calcium carbonate, a pore-former was added into the coacervation tank upon reaching the product temperature of approximately 58° C. while mixing to achieve a uniform distribution of the pore-former throughout the ethylcellulose membrane. Apart from this step, the procedure for making the microcapsules was unchanged.

2.B Ranitidine Hydrochloride ODTs, 150 mg: Orally disintegrating tablets lots (see Table 1 for compositions) were compressed into 13 mm tablets containing 150 mg equivalent ranitidine base, in accordance with the disclosures of Example 1.D above. Tablet lots (1135-027 and 1135-050) exhibited both acceptable taste-masking and dissolution at 45 minutes.

2.C Microencapsulation in 5-Gallon Solvent System: Ranitidine hydrochloride (Form II from Shasun Drugs and Chemicals meeting desired particle size/aspect ratio specifications) were fluid bed coated with an aqueous solution of Opadry Clear (hypromellose) at 4% solds in Glatt GPCG 1 (VersaGlatti) equipped with top spray for a weight gain of 2% w/w. Spraying conditions-Port size: 0.8 mm; atomization air pressure: 1.5 bar; bottom air distribution plate: granulation plate; inlet air temperature: 60° C.; product temperature: 51-54° C.; spray rate: 4 mL/min, and shake interval/time: 30 sec/3 sec. FIG. 2.B shows the micrographs of the ranitidine drug particles with said fluid bed coated first hydrophilic polymeric membrane. The fines adhering to larger drug particles appeared to be mostly wrapped up by the polymer membrane.

The fluid-bed coated drug particles (see Table 2 for compositions of Microcapsules) were charged into the 5-gallon system containing 10 kg of cyclohexane along with ethylcellulose (200 g) and polyethylene (200 g) and microencapsulated to produce microcapsules with said first hydrophilic polymeric membrane and said second coacervated polymeric membrane) in accordance with the disclosures disclosed above. Three lots of fluid bed coated ranitidine drug particles were microencapsulated with Ethocel and calcium carbonate at a ratio of 8:1 to 8:3 by adding/dispersing the micronized pore former at the tank temperature of about 58° C. during the cooling cycle. Ranitidine drug particles were also taste-masked without first applying said fluid bed coated hydrophilic polymeric membrane (lot 1135-CK-90).

TABLE 2
Compositions of Microcapsules of Ranitidine

| | | | | | Tablet Parameters | | |
|---|---|---|---|---|---|---|---|
| Microcapsule/ | Compositions (g) | | | EC coat/ | Weight | Hardness | Friability |
| Tablet Lot# | API | Ethocel | Ca CO₃ | Ratio* | (mg) | (N) | (%) |
| 1135-CK-090^{a} | 466.7 | 200 | | 30%/NA | 999.6 | 75 | 0.81 |
| 1135-CK-096 | 466.7 | 200 | | 30%/NA | 1025.6 | 71 | 0.99 |
| 1135-CK-099 | 245.5 | 200 | 50 | 45%/8:2 | 928.5 | 63 | 0.81 |
| 1135-CK-101 | 245.5 | 200 | 75 | 45%/8:3 | 967.1 | 71 | 0.80 |
| 1135-CK-103 | 245.5 | 200 | 25 | 45%/8:1 | 882.1 | 55 | 0.97 |
| 1135-CK-148** | 466.9 | 200 | | 30%/NA | 919 | 32 | 0.56 |

*→ Ratio of Ethocel to Calcium carbonate w/w
**→ ODT formulation contains microcrystalline cellulose (Avicel PH 101) at 10% by weight
^{a}→ First hydrophilic membrane not applied.

2.D Ranitidine ODTs, 150 mg: Taste-masked ranitidine drug particles (24-33% w/w), rapidly dispersing granules (67-54% w/w), a disintegrant (5% w/w), sweetener (0.1-0.5% w/w), flavor combination (0.5-3.5% w/w), MCC (0 or 10% w/w), and colorants (0.1-0.6% w/w) were blended together in a V blender and compressed into 150 mg (as ranitidine base). These tablets had acceptable organoleptic properties and dissolution profiles (see Table 2 for tableting properties and FIG. 3 for dissolution profiles).

### Example 3

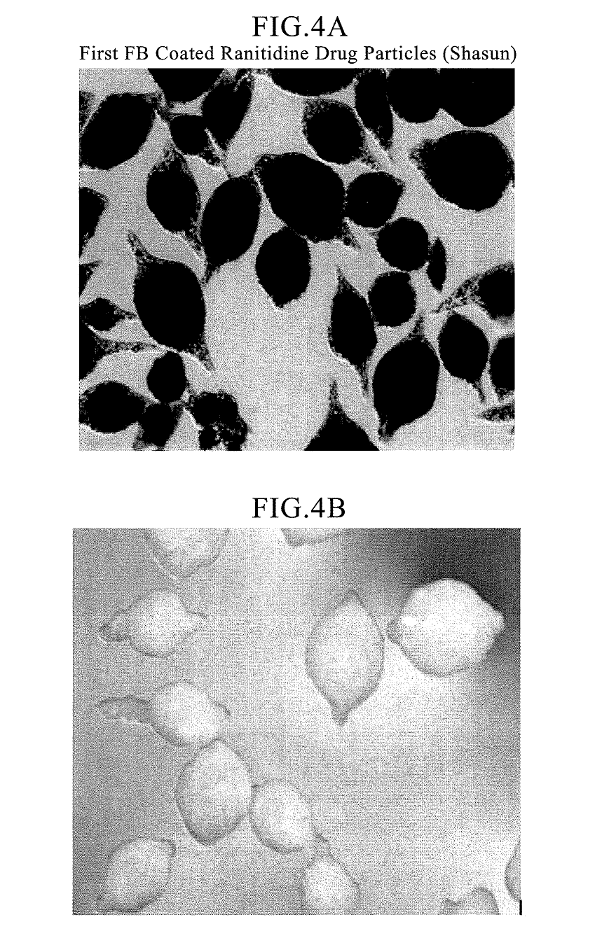

3.A First Polymeric Membrane by Coacervation: Ranitidine HCl (Form II) drug particles from Shasun (1100 g) were charged into the 5-gallon system along with Ethocel 100 cps (194.1 g) and Epolene (100 g) and microencapsulated for a coating of 15% by weight while mixing at the speed of 150 RPM in accordance with the disclosures above. Several lots of Microcapsules with said coacervated polymeric membrane were prepared under the same processing conditions. FIG. 4.A shows the micrographs of typical microcapsules produced by solvent coacervation.

3.B Second Water-insoluble-Gastrosoluble Polymeric Blend Membrane: Said coacervated drug particles from above were coated with a water-insoluble ethylcellulose (Ethocel Premium with a viscosity of 10 cps) and a gastrosoluble Eudragit E100 polymer (see Table 3 for compositions of the microcapsules) dissolved in 95/5 acetone/purified water in Glatt GPCG 3 equipped with a bottom spray Wurster insert under the following conditions:—port size: 1.0 mm; atomization air pressure: 2.3 bar; bottom air distribution plate: ‘C’ plate covered with 200 mesh product retention screen; inlet air temperature: 40±2° C.; product temperature: 30±2° C. (See FIG. 4B for the micrographs of the taste-masked (dual-coated) ranitidine particles).

TABLE 3
Compositions of Microcapsules and ODTs

| | Quantity per Batch (g) | | | |
|---|---|---|---|---|
| | | | | Flavor Layered |
| | Second FB Coated Microcapsules | | | Microcapsules |
| Ingredient | 1135-114 | 1135-143 | 1135-167 | 1309-025 |
| Coacervated | 900.00 | 1800.0 | 1500.0 | 1063.7 |
| Microcapsules | | | | |
| Ethylcellulose | 127.79 | 277.8 | 323.2 | 224.3 |
| Triethylcitrate | 12.75 | 27.72 | | 24.7 |
| Myvacet 9-45 | | | 40.5 | |
| Eudragit E100 | 127.79 | 277.8 | 323.2 | 198.4 |
| Talc | 7.67 | 16.7 | | |
| Magnesium Stearate | | | 45.5 | 30.4 |
| Acetone | 3233.8 | 5920.0 | 2707.6 | 1723.1 |
| Purified Water | 170.2 | 1480.0 | 1536.1 | 861.5 |
| Isopropyl Alcohol | | | 1340.1 | 861.5 |

3.C Flavor-coated Ranitidine Microcapsules: Coacervated ranitidine drug particles (1500 g) from Example 3A were charged into Glatt GPCG 3 equipped with a 7″ bottom spray Wurster insert and bottom air distribution plate ‘C’ covered with 200 mesh product retention screen. The desired flavor (50.1 g Cherry or Vanilla mint) and sucralose (13.57 g) were dissolved in purified water (214 g), and TEC (triethylcitrate) (16.6 g) was homogenized by mixing for 30 min. The flavor solution was sprayed at an increasing rate of 2 mL/min to 6 mL/min while maintaining a target product temperature of about 41° C. Following rinsing the spray system, fluid bed coating was continued by spraying EC-10/E100 solution at a rate of 6 mL/min increasing to 20 mL/min, inlet air volume set 53 cfm, atomization air pressure of 1.5 bar and target product temperature of 45° C. The coated drug particles were dried in the unit for 10 minutes to drive of excess residual solvents.

Following the procedures disclosed in Examples 3.A to 3.C, above, another batch (1385-019) of taste-masked ranitidine drug particles with the coacervation, flavor coating, and EC-10/EPO coating compositions identical to that of batch 1309-025 was also prepared

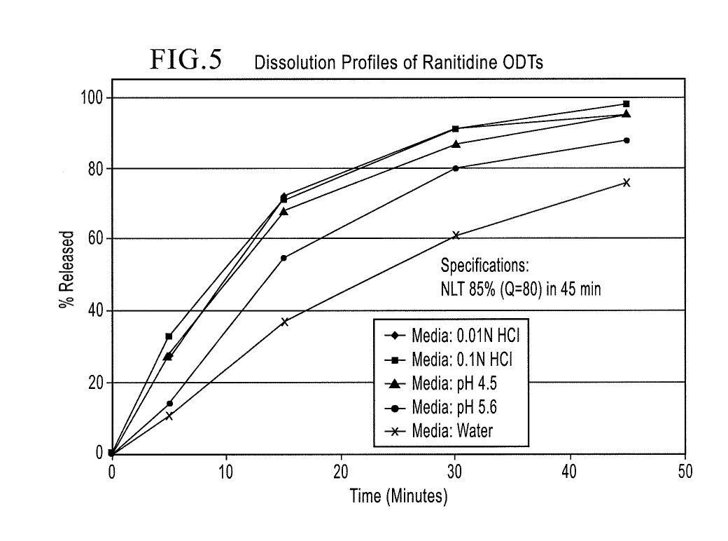

3.D ODTs containing Ranitidine Microcapsules: Appropriate amounts of said taste-masked rantitidine drug particles (lot #1135-143, 1135-167, 1309-025, or 1385-019) were blended with rapidly dispersing granules, and additional excipients (see Table 4 for the ingredients and compositions) in a V blender and compressed into 150 mg ODT tablets. These tablet lots exhibited acceptable organoleptic properties including aftertaste and met dissolution specification of NLT 85% in 45 minutes when dissolution tested in 900 mL of 0.1N HCl buffer. There are individuals, especially of Asian origin whose gastric pH is slightly less acidic compared to typical caucasians. For ODT lot# 1135-146, dissolution testing was performed in several media (e.g., 0.01N HCl, 0.1N HCl, pH 4.5, pH 5.6, as well as in purified water) to examine if the stomach pH under non-fasted conditions, and/or slightly less acidic gastric pH, would impact drug dissolution and hence therapeutic efficacy. The results of the dissolution testing (shown in FIG. 5) indicate that impact of gastric pH variations on drug dissolution is marginal. The ODT tablets containing additional flavor-sweetener coating on the drug particles had exceptionally good mouthfeel and aftertaste.

All patents, published patent applications, publications, and other documents cited herein are herein incorporated by reference in their entirety for all purposes.

TABLE 4
Compositions and Properties of ODTs of Ranitidine HCl

| | Orally Disintegrating Tablets - % per Tablet | | | | |
|---|---|---|---|---|---|
| | 1135-146 | 1135-177 | 1309-031 | 1309-037 | 1385-021 |
| Microcapsules (1135-143) | 27.00 | | | | |
| Microcapsules (1135-167) | | 29.35 | | | |
| Microcapsules (1309-025; | | | 27.97 | 27.97 | 29.54 |
| 1385-019) | | | | | |
| RD Granules | 54.30 | 51.45 | 52.84 | 54.88 | 36.27 |
| Crospovidone XL-10 | 5.0 | 5.0 | 5.0 | 5.0 | 10.00 |
| Microcryst. Cellulose | 10.0 | 10.0 | 10.0 | 10.0 | 20.00 |
| Sucralose | 0.20 | 0.35 | 0.35 | 0.35 | 0.35 |
| Vanilla Mint Combo | 3.35 | 3.70 | 3.65 | — | 3.65 |
| Cherry Flavor | | | | 1.30 | |
| Red/Yellow/Blue | 0.15 | 0.15 | 0.20 | 0.15 | 0.15 |
| Compression Force (kN) | 16 | 14 | 14 | 8 | 18 |
| Tablet Weight (mg) | 850.5 | 1011 | 1093 | 556 | 1065 |
| Hardness (N) | 33.5 | 51 | 59 | 46 | 56 |
| Friability (%) | 0.30 | 0.36 | 0.37 | 0.30 | 0.30 |
| Dissolution at 45 min (%) | 95 | | | | |

What is claimed is:

1. A pharmaceutical composition comprising a plurality of taste-masked microcapsules, comprising:
(a) a drug-containing core particle, comprising ranitidine and/or a pharmaceutically acceptable salt, solvate, or ester thereof,
(b) a first coating comprising a water-insoluble polymer disposed over said drug-containing core particle; and
(c) a second coating comprising a water-insoluble polymer and a gastrosoluble polymer disposed over said first coating.

2. The pharmaceutical composition of claim 1, wherein said first and/or second coating(s) substantially masks the taste of the ranitidine in the drug-containing core particle.

3. The pharmaceutical composition of claim 1, wherein said pharmaceutical composition releases at least about 70% of the total dose of ranitidine or a pharmaceutically acceptable salt, ester, solvate, or combination thereof, upon entering the stomach or within 30 minutes when tested for dissolution in simulated gastric fluid or 0.01 N HCl in United States Pharmacopoeia Apparatus 2 (paddles at 50 rpm in 900 mL of pH 1.2 buffer).

4. The pharmaceutical composition of claim 1, further comprising a sealant layer comprising a hydrophilic polymer disposed over said drug-containing core particle.

5. The pharmaceutical composition of claim 1, wherein the coating weights of said first and second coatings independently range from about 5% to about 40%.

6. The pharmaceutical composition of claim 1, wherein said drug-containing core particle comprises ranitidine hydrochloride.

7. The pharmaceutical composition of claim 1, wherein said drug-containing core particle comprises crystalline drug material, a drug granule, or a drug-layered bead.

8. The pharmaceutical composition of claim 7, wherein said drug-containing core particle comprises a crystalline drug material having an aspect ratio of not more than about 4.

9. The pharmaceutical composition of claim 6, wherein said drug-containing core particle is crystalline ranitidine hydrochloride.

10. The pharmaceutical composition of claim 1, wherein the coating weight of said first coating ranges from about 5% to about 20%.

11. The pharmaceutical composition of claim 1, wherein said water-insoluble polymer in said first coating is selected from the group consisting of ethylcellulose, polyvinyl acetate, cellulose acetate, cellulose acetate butyrate, neutral copolymers based on ethyl acrylate and methylmethacrylate, copolymers of acrylic and methacrylic acid esters with quaternary ammonium groups, and mixtures thereof.

12. The pharmaceutical composition of claim 1, wherein said water-insoluble polymer in said second coating is selected from the group consisting of ethylcellulose, cellulose acetate, cellulose acetate butyrate, polyvinyl acetate, neutral methacrylic acid-methylmethacrylate copolymers, neutral copolymers based on ethyl acrylate and methylmethacrylate, and mixtures thereof.

13. The pharmaceutical composition of claim 1, wherein said gastrosoluble polymer is selected from the group consisting of maltodextrins, aminoalkyl methacrylate copolymers, polyvinylacetate diethaminoacetate, and combinations thereof.

14. The pharmaceutical composition of claim 1, wherein in said second coating the ratio of said water-insoluble polymer to said gastrosoluble polymer ranges from about 95:5 to about 50:50.

15. The pharmaceutical composition of claim 1, wherein said microcapsules have an average particle size of less than about 500 μm.

16. The pharmaceutical composition of claim 1, further comprising a sealant layer which comprises a hydrophilic polymer, disposed over said drug-containing core particle and under said first and second coatings.

17. The pharmaceutical composition of claim 16, wherein said hydrophilic polymer is selected from the group consisting of hydroxypropyl methylcellulose, hydroxypropylcellulose, and polyvinylpyrrolidone.

18. The pharmaceutical composition of claim 1, further comprising a third coating which comprises a flavor and/or sweetener.

19. The pharmaceutical composition of clam 18, wherein said third coating is disposed between said first and second coatings.

20. The pharmaceutical composition of claim 18, wherein said third coating has a coating weight ranging from about 2% to about 10%.

21. The pharmaceutical composition of claim 18, wherein at least one of said first, second, and third coatings further comprise a plasticizer.

22. The pharmaceutical composition of claim 21, wherein in each coating in which the plasticizer is present, said plasticizer is independently selected from the group consisting of polyethylene glycols, triacetin, tributyl citrate, triethyl citrate, acetyl tri-n-butyl citrate, diethyl phthalate, castor oil, dibutyl sebacate, monoacetylated glycerides, acetylated monoglycerides, acetylated diglycerides, and mixtures thereof.

23. A pharmaceutical dosage form comprising:
a) the pharmaceutical composition of claim 1; and
b) rapidly dispersing granules comprising a saccharide and/or sugar alcohol in combination with a disintegrant,
wherein said pharmaceutical dosage form is an orally disintegrating tablet.

24. The pharmaceutical dosage form of claim 23, wherein said disintegrant is selected from the group consisting of crospovidone, sodium starch glycolate, crosslinked sodium carboxymethyl cellulose, and low-substituted hydroxypropylcellulose, and said saccharide and/or sugar alcohol is selected from the group consisting of sucralose, lactose, sucrose, maltose, mannitol, sorbitol, xylitol, and maltitol.

25. The pharmaceutical dosage form of claim 23, wherein the ratio of said disintegrant to said saccharide and/or sugar alcohol ranges from about 10:90 to about 1:99.

26. The pharmaceutical dosage form of claim 23, wherein said disintegrant and said sugar alcohol and/or said saccharide are each present in the form of microparticles having an average particle size of about 30 μm or less.

27. The pharmaceutical dosage form of claim 23, wherein the ratio of said taste-masked microcapsules to said rapidly disintegrating granules ranges from about 1:6 to about 1:1.

28. The pharmaceutical dosage form of claim 23, wherein said orally disintegrating tablet substantially disintegrates within about 60 seconds after contact with saliva in the oral cavity or simulated saliva fluid, or within about 60 seconds when tested by the <USP 701> Disintegration Test.

29. The pharmaceutical dosage form of claim 23, wherein said orally disintegrating tablet has a friability of less than about 1%, and a mean hardness value of from about 20 N to about 80 N.

30. A method for the preparation of the pharmaceutical composition of claim 1, comprising:
a) coating said drug-containing core particles with a first solution comprising a water-insoluble polymer and a solvent, thereby forming coated drug particles; and
b) coating said coated drug particles formed in step (a) with a second solution comprising a water-insoluble polymer and a gastrosoluble polymer, thereby forming said taste-masked microcapsules.

31. The method of claim 30, wherein said coating of step (a) is by coacervation.

32. The method of claim 30, further comprising
(c) coating said coated drug-containing core particles or said taste-masked microcapsules formed in step (b), with a third solution comprising one or more flavoring agents or sweeteners.

33. A method of preparing an orally disintegrating tablet comprising said taste-masked microcapsules of claim 1, comprising:
a) mixing said taste-masked microcapsules with rapidly dispersing granules comprising a saccharide and/or sugar alcohol in combination with a disintegrant, thereby forming a compression blend; and
b) compressing said compression blend into an orally disintegrating tablet.

34. A method of treating or preventing a gastrointestinal disorder comprising administering to a patient the pharmaceutical composition of claim 1.

35. A pharmaceutical composition comprising a plurality of taste-masked microcapsules, comprising:
(a) a drug-containing core particle, comprising ranitidine and/or a pharmaceutically acceptable salt or ester thereof;
(b) a first coating comprising a water-insoluble polymer and a gastrosoluble pore-former disposed over said drug-containing core particle; and
(c) a second coating comprising a flavor and/or sweetener disposed over said drug-containing core particle.

36. The pharmaceutical composition of claim 35, wherein said first and second coatings substantially mask the taste of the ranitidine in the drug-containing core particle.

37. The pharmaceutical composition of claim 35, wherein said pharmaceutical composition releases at least about 70% of the total dose of ranitidine or a pharmaceutically acceptable salt, ester, solvate, or combination thereof upon entering the stomach, or within 30 minutes when tested for dissolution in simulated gastric fluid or 0.01 N HCl in United States Pharmacopoeia Apparatus 2 (paddles at 50 rpm in 900 mL of pH 1.2 buffer).

38. The pharmaceutical composition of claim 35, further comprising a sealant layer comprising a hydrophilic polymer disposed over said drug-containing core particle.

39. The pharmaceutical composition of claim 35, wherein the coating weights of said first and second coatings independently range from about 5% to about 40%.

40. The pharmaceutical composition of claim 35, wherein said drug-containing core particle comprises ranitidine hydrochloride.

41. The pharmaceutical composition of claim 35, wherein said drug-containing core particle comprises crystalline drug material, a drug granule, or a drug-layered bead.

42. The pharmaceutical composition of claim 41, wherein said drug-containing core particle comprises a crystalline drug material having an aspect ratio of not more than about 4.

43. The pharmaceutical composition of claim 40, wherein said drug-containing core particle is crystalline ranitidine hydrochloride.

44. The pharmaceutical composition of claim 35, wherein the coating weight of said first coating ranges from about 20% to about 40%.

45. The pharmaceutical composition of claim 35, wherein said water-insoluble polymer is selected from the group consisting of ethylcellulose, cellulose acetate, cellulose acetate butyrate, polymethacrylates, carboxymethyl ethylcellulose, polylactic acid, and mixtures thereof.

46. The pharmaceutical composition of claim 35, wherein said gastrosoluble pore-former is selected from the group consisting of maltodextrins, aminoalkyl methacrylate copolymers, polyvinylacetate diethaminoacetate, and combinations thereof.

47. The pharmaceutical composition of claim 35, wherein said gastrosoluble pore-former is selected from the group consisting of calcium carbonate, calcium phosphate, calcium saccharide, calcium succinate, calcium tartrate, ferric acetate, ferric hydroxide, ferric phosphate, magnesium carbonate, magnesium citrate, magnesium hydroxide, magnesium phosphate, and a mixture thereof.

48. The pharmaceutical composition of claim 35, wherein the ratio of said water-insoluble polymer to said gastrosoluble pore-former in said first coating ranges from about 95:5 to about 50:50.

49. The pharmaceutical composition of claim 35, wherein said microcapsules have an average particle size of less than about 500 μm.

50. The pharmaceutical composition of claim 35, further comprising a sealant layer which comprises a hydrophilic polymer, disposed over said drug-containing core particle and under said first and second coatings.

51. The pharmaceutical composition of claim 50, wherein said hydrophilic polymer is selected from the group consisting of hydroxypropyl methylcellulose, hydroxypropylcellulose, and polyvinylpyrrolidone.

52. The pharmaceutical composition of claim 35, wherein said second coating has a coating weight ranging from about 2% to about 10%.

53. The pharmaceutical composition of claim 35, wherein at least one of said first and second coatings further comprises a plasticizer.

54. The pharmaceutical composition of claim 53, wherein in each coating where the plasticizer is present, said plasticizer is independently selected from the group consisting of polyethylene glycols, triacetin, tributyl citrate, triethyl citrate, acetyl tri-n-butyl citrate, diethyl phthalate, castor oil, dibutyl sebacate, monoacetylated glycerides, acetylated monoglycerides, acetylated diglycerides, and mixtures thereof.

55. A pharmaceutical dosage form comprising:
a) the pharmaceutical composition of claim 35; and
b) rapidly dispersing granules comprising a saccharide and/or sugar alcohol in combination with a disintegrant,
wherein said pharmaceutical dosage form is an orally disintegrating tablet.

56. The pharmaceutical dosage form of claim 55, wherein said disintegrant is selected from the group consisting of crospovidone, sodium starch glycolate, crosslinked sodium carboxymethyl cellulose, and low-substituted hydroxypropylcellulose, and said saccharide and/or sugar alcohol is selected from the group consisting of sucralose, lactose, sucrose, maltose, mannitol, sorbitol, xylitol, and maltitol.

57. The pharmaceutical dosage form of claim 55, wherein the ratio of said disintegrant to said saccharide and/or sugar alcohol ranges from about 10:90 to about 1:99.

58. The pharmaceutical dosage form of claim 55, wherein said disintegrant and said sugar alcohol and/or said saccharide are each present in the form of microparticles having an average particle size of about 30 μm or less.

59. The pharmaceutical dosage form of claim 55, wherein the ratio of said taste-masked microcapsules to said rapidly disintegrating granules ranges from about 1:6 to about 1:1.

60. The pharmaceutical dosage form of claim 55, wherein said orally disintegrating tablet substantially disintegrates within about 60 seconds after contact with saliva in the oral cavity or simulated saliva fluid, or within about 60 seconds when tested by the <USP 701> Disintegration Test.

61. The pharmaceutical dosage form of claim 55, wherein said orally disintegrating tablet has a friability of less than about 1%, and a mean hardness value of from about 20 N to about 80 N.

62. A method for the preparation of the pharmaceutical composition of claim 35, comprising:
a) coating said drug-containing core particles with a first solution comprising a water-insoluble polymer, a gastrosoluble pore-former, and a solvent, thereby forming coated drug particles; and
c) coating said coated drug particles with a second solution comprising one or more flavoring agents or sweeteners and a solvent, thereby forming said taste-masked microcapsules.

63. The method of claim 62, further comprising:
a1) coating said drug-containing core particles with a hydrophilic polymer.

64. A method of preparing an orally disintegrating tablet comprising:
a) mixing said taste-masked microcapsules of claim 35 with rapidly dispersing granules comprising a saccharide and/or sugar alcohol in combination with a disintegrant, thereby forming a compression blend; and
b) compressing said compression blend into an orally disintegrating tablet.

65. A method of treating or preventing a gastrointestinal disorder comprising administering to a patient the pharmaceutical composition of claim 35.
